(19) United States
(12) Patent Application Publication
(10) Pub. No.: US 2013/0338129 A1
(43) Pub. Date: Dec. 19, 2013

(54) METHOD OF TREATING REFRACTORY CANCER

(71) Applicant: Niiki Pharma Inc., Tampa, FL (US)

(72) Inventor: Hooshmand Sheshbaradaran, Hoboken, NJ (US)

(73) Assignee: Niiki Pharma Inc., Tampa, FL (US)

(21) Appl. No.: 13/974,958

(22) Filed: Aug. 23, 2013

Related U.S. Application Data
(63) Continuation of application No. 13/743,356, filed on Jan. 17, 2013.
Application No. 13/743,356 is a continuation of application No. PCT/US2011/044301, filed on Jul. 17, 2011.
(60) Provisional application No. 61/365,328, filed on Jul. 17, 2010.

(51) Int. Cl. A61K 31/555

(52) U.S. Cl. CPC A61K 31/555
USPC 514/186

(57) ABSTRACT

Therapeutic methods for treating refractory cancers are disclosed comprising administering to a patient in need of treatment a ruthenium complex salt.

### RELATED APPLICATIONS

This application is a continuation U.S. patent application Ser. No. 13/743,356 filed on Jan. 17, 2013, which is a continuation of PCT/US2011/044301 filed Jul. 17, 2011, which claims the priority of U.S. Provisional Application No. 61/365,328 filed on Jul. 17, 2010, the entirety of each is hereby incorporated herein by reference.

### FIELD OF THE INVENTION

The present invention generally relates to methods for treating cancer, and particularly to a method of treating refractory cancer.

### BACKGROUND OF THE INVENTION

A number of ruthenium complex compounds are known in the art to be useful as anti-tumor compounds. See e.g., U.S. Pat. No. 4,843,069 PCT Publication No. WO 9736595, and US Application Publication No. 2005032801. In particular, the ruthenium complex salts indazolium trans-[tetrachlorobis(1H-indazole)ruthenate (III)] and sodium trans-[tetrachlorobis(1H-indazole)ruthenate (III)] have been shown in preclinical studies to be effective in inducing apoptosis in colon cancer cells. In addition, the compound ruthenium complex salt indazolium trans-[tetrachlorobis(1H-indazole)ruthenate (III)] (KP1019) showed some anti-cancer activities in a phase I clinical trial.

### SUMMARY OF THE INVENTION

It has now been discovered that the compound sodium trans-[tetrachlorobis(1H-indazole)ruthenate(III)] is especially effective in treating certain refractory cancers. Specifically, it has been surprisingly discovered in a clinical study that the compound sodium trans-[tetrachlorobis(1H-indazole)ruthenate(III)] is effective in controlling colorectal cancer that had failed oxaliplatin, capecitibine, cetuximab, as well as irinotecan and panitumumab. The compound is also effective in temozolomide-resistant melanoma cells. In addition, it has been discovered that the compound is able to control refractory lung cancers.

Accordingly, in a first aspect, the present invention provides a method of treating refractory colorectal cancer, which comprises treating a patient identified as having colorectal cancer refractory to a treatment including one or more of the group of oxaliplatin, capecitibine, cetuximab, irinotecan and panitumumab, with a therapeutically effective amount of sodium trans-[tetrachlorobis(1H-indazole)ruthenate(III)].

In another aspect, the present invention provides a method of treating melanoma refractory to temozolomide, which comprises identifying a patient having melanoma refractory to temozolomide and treating the patient with a therapeutically effective amount of sodium trans-[tetrachlorobis(1H-indazole)ruthenate(III)].

The present invention further provides a method of treating refractory lung cancer such as non-small cell lung cancer (NSCLC), which comprises identifying a patient having such a refractory lung cancer and treating the patient with a therapeutically effective amount of sodium trans-[tetrachlorobis(1H-indazole)ruthenate(III)]. In some embodiments, the refractory lung cancer is refractory to a treatment comprising paclitaxel. In some embodiments, the refractory lung cancer is NSCLC resistant to EGFR inhibitors such as erlotinib and gefitinib, or having NSCLC cells having the T790M mutation in the EGFR gene. In some embodiments, the refractory lung cancer has been previously treated with a regimen comprising one or more drugs chosen from carboplatin, gemcitabine, zoledronic acid, pemetrexed, gemcitabine, navelbine, vatalanib, imatinib, and bevacizumab.

The foregoing and other advantages and features of the invention, and the manner in which the same are accomplished, will become more readily apparent upon consideration of the following detailed description of the invention taken in conjunction with the accompanying examples, which illustrate preferred and exemplary embodiments.

### BRIEF DESCRIPTION OF THE DRAWINGS

FIG. 1 is a sigmoidal dose response curve from an MTT assay of G361 cells treated with sodium trans-[tetrachlorobis(1H-indazole)ruthenate(III)] (Y axis: Percent of Control; X axis: Concentration); and

FIG. 2 is a sigmoidal dose response curve from an MTT assay of G361 cells treated with temozolomide (Y axis: Percent of Control. X axis: Concentration).

### DETAILED DESCRIPTION OF THE INVENTION

The present invention generally provides methods for treating specific refractory cancers. The term “refractory to (a treatment),” as used herein, means that a particular cancer either fails to respond favorably to a specific anti-neoplastic treatment, or alternatively, recurs or relapses after responding favorably to a specific anti-neoplastic treatment. Accordingly, for example, a non-small cell lung cancer “refractory to” erlotinib means that a non-small cell lung cancer either has failed to respond favorably to, or is resistant to, a treatment regimen that includes, but not necessarily limited to, erlotinib, or alternatively, has recurred or relapsed after responding favorably to the treatment regimen.

To detect or identify a refractory cancer, patients undergoing a chemotherapy treatment can be carefully monitored for signs of resistance, non-responsiveness or recurring cancer. This can be accomplished by monitoring the patient's cancer's response to the chemotherapy treatment. The response, lack of response, or relapse of the cancer to the initial treatment can be determined by any suitable method practiced in the art. For example, this can be accomplished by the assessment of tumor size and number. An increase in tumor size or, alternatively, tumor number, indicates that the tumor is not responding to the chemotherapy, or that a relapse has occurred. The determination can be done according to the “RECIST” criteria as described in detail in Therasse et al, J. Natl. Cancer Inst., 92:205-216 (2000).

In accordance with a first aspect of the present invention, a method is provided for treating colorectal cancer previously treated with a regimen including one, two, three or more drugs selected from the group consisting of oxaliplatin, capecitibine, cetuximab, irinotecan and panitumumab. The method can be useful in treating and preventing refractory colorectal cancer, or delaying the recurrence of colorectal cancer. The method comprises administering to a patient identified as having such previously treated colorectal cancer, a therapeutically effective amount of an alkali metal salt of trans-[tetrachlorobis(1H-indazole)ruthenate(III)], in particular, sodium trans-[tetrachlorobis(1H-indazole)ruthenate(III)]. In some embodiments, the method is applied to treat a patient having colorectal cancer refractory to a treatment regimen including one or more drugs selected from the group consisting of oxaliplatin, capecitibine, cetuximab, irinotecan and panitumumab, by administering to the patient a therapeutically effective amount of an alkali metal salt of trans-[tetrachlorobis(1H-indazole)ruthenate(III)], in particular, sodium trans-[tetrachlorobis(1H-indazole)ruthenate(III)]. That is, the present invention is directed to the use of an alkali metal salt of trans-[tetrachlorobis(1H-indazole)ruthenate(III)], in particular, sodium trans-[tetrachlorobis(1H-indazole)ruthenate(III)], for the manufacture of a medicament for treating colorectal cancer previously treated with, or refractory to, a treatment regimen comprising one, two, three, or more drugs chosen from the group consisting of oxaliplatin, capecitibine, cetuximab, irinotecan and panitumumab.

In some embodiments, the method comprises administering an alkali metal salt of trans-[tetrachlorobis(1H-indazole)ruthenate(III)], in particular, sodium trans-[tetrachlorobis(1H-indazole)ruthenate(III)] to a patient having colorectal cancer previously treated with a regimen comprising oxaliplatin. In some embodiments, the method comprises administering an alkali metal salt of trans-[tetrachlorobis(1H-indazole)ruthenate(III)], in particular, sodium trans-[tetrachlorobis(1H-indazole)ruthenate(III)] to a patient having colorectal cancer previously treated with a regimen comprising irinotecan. In some embodiments, a therapeutically effective amount of an alkali metal salt of trans-[tetrachlorobis(1H-indazole)ruthenate(III)], in particular, sodium trans-[tetrachlorobis(1H-indazole)ruthenate(III)] is administered to a patient having colorectal cancer previously treated with the FOLFOX regimen (folinic acid, 5-fluorouracil, and oxaliplatin), the FOLFIRI regimen (folinic acid, 5-fluorouracil & irinotecan), a regimen including oxaliplatin and capecitibine, or a regimen including irinotecan, each with or without bevacizumab, or alternatively, each with or without an EGFR antibody (e.g., cetuximab and panitumumab). In another embodiment, a therapeutically effective amount of an alkali metal salt of trans-[tetrachlorobis(1H-indazole)ruthenate(III)], in particular, sodium trans-[tetrachlorobis(1H-indazole)ruthenate(III)] is administered to a patient having colorectal cancer previously treated with, or refractory to, capecitabine and one or more other drugs. The refractory colorectal cancer can be at any stage, either local or metastatic.

In another aspect, the present invention provides a method for treating melanoma previously treated with temozolomide. Thus, the method is useful in treating and preventing refractory melanoma, or delaying the recurrence of melanoma previously treated with temozolomide. Specifically, the method comprises administering to a patient having melanoma previously treated with temozolomide, a therapeutically effective amount of an alkali metal salt of trans-[tetrachlorobis(1H-indazole)ruthenate(III)], in particular, sodium trans-[tetrachlorobis(1H-indazole)ruthenate(III)]. That is, the invention is directed to the use of an alkali metal salt of trans-[tetrachlorobis(1H-indazole)ruthenate(III)], in particular, sodium trans-[tetrachlorobis(1H-indazole)ruthenate(III)], for the manufacture of a medicament for treating and preventing melanoma refractory to a treatment regimen comprising temozolomide. In some embodiments, a melanoma patient refractory to a treatment regimen comprising temozolomide is identified, and a therapeutically effective amount of an alkali metal salt of trans-[tetrachlorobis(1H-indazole)ruthenate(III)] such as sodium trans-[tetrachlorobis(1H-indazole)ruthenate(III)] is administered to the patient.

In yet another aspect, the present invention provides a method for treating and preventing refractory lung cancer, particularly non-small cell lung cancer (NSCLC), or delaying the recurrence of lung cancer such as NSCLC. Specifically, the method comprises administering a therapeutically effective amount of an alkali metal salt of trans-[tetrachlorobis(1H-indazole)ruthenate(III)], in particular, sodium trans-[tetrachlorobis(1H-indazole)ruthenate(III)] to a patient having lung cancer, particularly NSCLC, (1) having cells harboring the T790M mutation in the EGFR gene or (2) previously treated with, e.g., refractory to or resistant to, a regimen comprising one, two, three or more drugs chosen from the group of paclitaxel, docetaxel, carboplatin, bevacizumab, sorafenib, gemcitabine, zoledronic acid, pemetrexed, navelbine, vatalanib, imatinib, and EGFR inhibitors (e.g., erlotinib, gefitinib, cetuximab and panutimumab). That is, the invention is directed to the use of an alkali metal salt of trans-[tetrachlorobis(1H-indazole)ruthenate(III)], in particular, sodium trans-[tetrachlorobis(1H-indazole)ruthenate(III)], for the manufacture of a medicament for treating and preventing refractory lung cancer, particularly non-small cell lung cancer (NSCLC), having the T790M mutation in the EGFR gene, or previously treated with, e.g., refractory to or resistant to, one, two, three or more drugs chosen from the group consisting of paclitaxel, docetaxel, carboplatin, bevacizumab, sorafenib, gemcitabine, zoledronic acid, pemetrexed, navelbine, vatalanib, imatinib, and EGFR inhibitors (e.g., erlotinib, gefitinib, cetuximab and panutimumab).

In one embodiment, the method comprises determining if a NSCLC patient has the T790M mutation in the EGFR gene in the tumor cells, and if the mutation is identified, administering to the patient a therapeutically effective amount of an alkali metal salt of trans-[tetrachlorobis(1H-indazole)ruthenate(III)], in particular, sodium trans-[tetrachlorobis(1H-indazole)ruthenate(III)].

In another embodiment, the method comprises administering to a patient previously treated with a regimen comprising paclitaxel, e.g., refractory to or resistant to paclitaxel, a therapeutically effective amount of an alkali metal salt of trans-[tetrachlorobis(1H-indazole)ruthenate(III)], in particular, sodium trans-[tetrachlorobis(1H-indazole)ruthenate(III)].

In another embodiment, the method comprises administering to a lung cancer patient previously treated with a regimen comprising an EGFR inhibitor (e.g., erlotinib, gefitinib), e.g., refractory to or resistant to erlotinib or gefitinib, a therapeutically effective amount of an alkali metal salt of trans-[tetrachlorobis(1H-indazole)ruthenate(III)], in particular, sodium trans-[tetrachlorobis(1H-indazole)ruthenate(III)].

In another embodiment, the method comprises administering to a lung cancer patient previously treated with a regimen comprising gemcitabine, e.g., refractory to or resistant to gemcitabine, a therapeutically effective amount of an alkali metal salt of trans-[tetrachlorobis(1H-indazole)ruthenate(III)], in particular, sodium trans-[tetrachlorobis(1H-indazole)ruthenate(III)].

In another embodiment, the method comprises administering to a lung cancer patient previously treated with a regimen comprising one, two or three drugs selected from the group of docetaxel, carboplatin and bevacizumab, e.g., refractory to or resistant to such a regimen, a therapeutically effective amount of an alkali metal salt of Trans-[tetrachlorobis(1H-indazole)ruthenate(III)], in particular, sodium trans-[tetrachlorobis(1H-indazole)ruthenate(III)].

In another embodiment, the method comprises administering to a lung cancer patient previously treated with a regimen comprising sorafenib, e.g., refractory to or resistant to sorafenib, a therapeutically effective amount of an alkali metal salt of trans-[tetrachlorobis(1H-indazole)ruthenate(III)], in particular, sodium trans-[tetrachlorobis(1H-indazole)ruthenate(III)].

In yet another embodiment, the method comprises administering to a lung cancer patient previously treated with a regimen comprising pemetrexed, e.g., refractory to or resistant to pemetrexed, a therapeutically effective amount of an alkali metal salt of trans-[tetrachlorobis(1H-indazole)ruthenate(III)], in particular, sodium trans-[tetrachlorobis(1H-indazole)ruthenate(III)].

In yet another embodiment, the method comprises administering to a lung cancer patient previously treated with a regimen comprising one, two or three drugs chosen from the group of carboplatin, gemcitibine and zoledronic acid, e.g., refractory to or resistant to such a regimen, an effective amount of an alkali metal salt of trans-[tetrachlorobis(1H-indazole)ruthenate(III)], in particular, sodium trans-[tetrachlorobis(1H-indazole)ruthenate(III)].

In yet another embodiment, the method comprises administering to a lung cancer patient previously treated with a regimen comprising bevacizumab, e.g., refractory to or resistant to such a regimen, an effective amount of an alkali metal salt of trans-[tetrachlorobis(1H-indazole)ruthenate(III)], in particular, sodium trans-[tetrachlorobis(1H-indazole)ruthenate(III)].

In some embodiments, the lung cancer is small cell lung cancer. In preferred embodiments, the lung cancer is NSCLC such as lung adenocarcinoma and squamous cell lung cancer.

In the various aspects described above, the methods may optionally further include a step of identifying a patient having a refractory cancer as described, beside the administering step.

For purposes of preventing or delaying cancer recurrence, cancer patients who have been treated and are in remission or in a stable or progression free state may be treated with a prophylatically effective amount of an alkali metal salt of trans-[tetrachlorobis(1H-indazole)ruthenate(III)], particularly sodium trans-[tetrachlorobis(1H-indazole)ruthenate(III)] to effectively prevent or delay the recurrence or relapse of the cancer.

As used herein, the phrase “treating . . . with . . .” or a paraphrase thereof means administering a compound to the patient or causing the formation of a compound inside the body of the patient.

In accordance with the method of the present invention, a therapeutically effective amount of an alkali metal salt of trans-[tetrachlorobis(1H-indazole)ruthenate(III)], in particular sodium trans-[tetrachlorobis(1H-indazole)ruthenate(III)] can be used alone, or alternatively in combination with one or more other anti-cancer agents.

Alkali metal salts of trans-[tetrachlorobis(1H-indazole)ruthenate(III)] can be made in any methods known in the art. For example, PCT Publication No. WO/2008/154553 discloses an efficient method of making sodium trans-[tetrachlorobis(1H-indazole)ruthenate(III)].

The pharmaceutical compounds such as sodium trans-[tetrachlorobis(1H-indazole)ruthenate(III)] can be administered through intravenous injection or any other suitable means at an amount of from 0.1 mg to 1000 mg per kg of body weight of the patient based on total body weight. The active ingredients may be administered at once, or may be divided into a number of smaller doses to be administered at predetermined intervals of time, e.g., once daily or once every two days. In preferred embodiments, sodium trans-[tetrachlorobis(1H-indazole)ruthenate(III)] is administered intravenously at 320 mg/m² or 500 mg/m² or greater. It can be administered, for example, once a week, e.g., on day 1, day 8 and day 15 of each 28-day cycle. It should be understood that the dosage ranges set forth above are exemplary only and are not intended to limit the scope of this invention. The therapeutically effective amount of the active compound can vary with factors including, but not limited to, the activity of the compound used, stability of the active compound in the patient's body, the severity of the conditions to be alleviated, the total weight of the patient treated, the route of administration, the ease of absorption, distribution, and excretion of the active compound by the body, the age and sensitivity of the patient to be treated, and the like. The amount of administration can be adjusted as the various factors change over time.

In some embodiments, a pharmaceutically acceptable salt (e.g., an alkali metal salt preferably sodium trans-[tetrachlorobis(1H-indazole)ruthenate(III)]) is administered to a patient at an amount of at least 300, 320, 400, 500, 550, 600, 650, 700, 800 mg/m² or greater based on body surface area, at each administration. In some embodiments, a pharmaceutically acceptable salt (e.g., an alkali metal salt preferably sodium trans-[tetrachlorobis(1H-indazole)ruthenate(III)]) is administered to a patient by at an amount of greater than 500 mg, 600 mg, 700 mg, 800 mg, 900 mg or 1000 mg at each administration. In preferred embodiments, the drug is administered by intravenous injection once per week, on days 1, 8, and 15 of each 28-day cycle.

In accordance with the present invention, an alkali metal salt of trans-[tetrachlorobis(1H-indazole)ruthenate(III)] (e.g., sodium trans-[tetrachlorobis(1H-indazole)ruthenate(III)]) can be incorporated into a drug product, e.g., in an injectable form suitable for intravenous, intra-arterial, intradermal, or intramuscular administration. Injectable forms are generally known in the art, e.g., in buffered solution or suspension.

In accordance with another aspect of the present invention, a pharmaceutical kit is provided comprising in a container a unit dosage form of an alkali metal salt of trans-[tetrachlorobis(1H-indazole)ruthenate(III)] (e.g., sodium trans-[tetrachlorobis(1H-indazole)ruthenate(III)]), and optionally instructions for using the kit in the methods in accordance with the present invention, e.g., treating, preventing or delaying the onset of refractory cancer, as described above. The amount of a therapeutic compound in the unit dosage form is determined by the dosage to be used on a patient in the methods of the present invention. In the kit, an alkali metal salt of trans-[tetrachlorobis(1H-indazole)ruthenate(III)] (e.g., sodium trans-[tetrachlorobis(1H-indazole)ruthenate(III)]) can be in lyophilized form in an amount of, e.g., 25 mg, in an ampoule. In the clinic, the lyophilized form can be dissolved and administered to a patient in need of the treatment in accordance with the present invention.

### EXAMPLES

1. In Vivo Activities of Sodium Trans-[tetrachlorobis(1H-indazole)ruthenate(III)] in Refractory Colon Cancer

A human clinical trial was conducted in two centers in the United Kingdom (UK) with sodium trans-[tetrachlorobis(1H-indazole)ruthenate(III)]. The trial enrolled a 63 year old white male with colorectal adenocarcinoma, adenocystic histology, incompletely resected in September 2007. His initial therapy consisted of oxaliplatin, capecitibine and cetuximab between October 2007 and February 2009 with best response of progressive disease. That is, the therapy failed to control the growth of the tumors. He then was changed to irinotecan and panitumumab, administered between April 2009 and October 2009, again with best response progressive disease. Beginning in June 2010, he was placed on sodium trans-[tetrachlorobis(1H-indazole)ruthenate(III)] as a single agent administered intravenously at 500 mg/m² (based on body surface area, i.e., BSA) daily on days 1 through 4 of each 21-day cycle. The patient received 4 cycles of sodium trans-[tetrachlorobis(1H-indazole)ruthenate(III)] with best response of stable disease. Thus, sodium trans-[tetrachlorobis(1H-indazole)ruthenate(III)] was able to control colorectal cancer that had failed oxaliplatin, capecitibine and cetuximab, as well as irinotecan and panitumumab.

2. Activities of Sodium Trans-[tetrachlorobis(1H-indazole)ruthenate(III)] in Temozolomide-Resistant Melanoma Cells and Paclitaxel- and Erlotinib-Resistant Lung Cancer Cells

The anti-proliferative activities of sodium trans-[tetrachlorobis(1H-indazole)ruthenate(III)], against the indicated cell lines were evaluated in vitro using the ATCC's MTT Cell Proliferation Assay (Catalog No. 30-1010K). Human malignant melanoma cell line G361 plates were seeded with 2,500 cells/well, and the cells were grown in McCoy's 5a medium containing 10% FBS and 1% penicillin/strep/glutamine. Human lung carcinoma cell line A549 plates were seeded with 2,500 cells/well, and the cells were grown in Ham's F12 medium containing 10% FBS and 1% penicillin/strep/glutamine. Cultures were maintained in a 37° C. humidified 5% CO₂/95% air atmosphere. Stock cultures were allowed to grow to 70-80% confluence for this study. The cells were treated with sodium trans-[tetrachlorobis(1H-indazole)ruthenate(III)] or temozolomide at 1,000 μM, or a series of 4× dilutions thereof (250 μM, 62.5 μM, etc.), or with paclitaxel at 1000 nM, or a series of 4× dilutions thereof. 1000 μl of medium was removed from each well at 72 hours post-treatment and 10 μl MTT reagent was added to each well. The plates were incubated at 37° C. for 4 hours and then 100 μl of detergent was added. The plates were left overnight at room temperature in the dark and was read on a plate reader using SoftMax® Pro (version 5.2, Molecular Devices).

The absorbance data was analyzed as follows: Absorbance values were converted to Percent of Control and plotted against test agent concentrations for IC₅₀ calculations using SoftMax® Pro (version 5.2, Molecular Devices). The plate blank signal average was subtracted from all wells prior to calculating the Percent of Control. Percent of Control values were calculated by dividing the absorbance values for each test well by the No Drug Control average (column 11 values; cells+vehicle control) and multiplying by 100. Plots of Compound Concentration versus Percent of Control were analyzed using the 4-parameter equation to obtain IC₅₀ values and other parameters that describe the sigmoidal dose response curve.

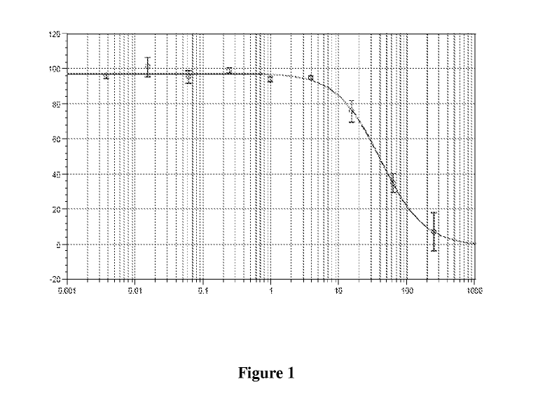

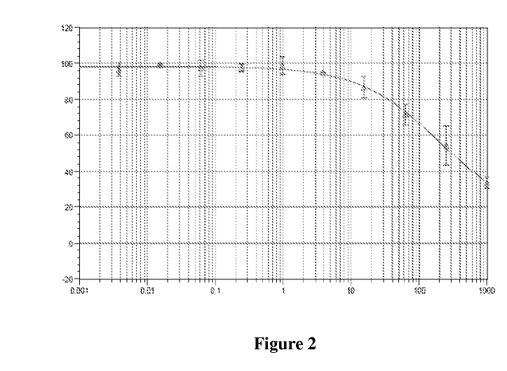

The IC₅₀ values for the test agents were estimated by curve-fitting the data using the following four parameter-logistic equation:

$$Y = \frac{Top - Bottom}{1 + {( \frac{X}{{IC}_{50}} )}^{n}} + Bottom$$

wherein “Top” is the maximal % of control absorbance (100%), “Bottom” is the minimal % of control absorbance at the highest agent concentration (down to zero), Y is the Percent of Control absorbance, X is the test agent Concentration, IC₅₀ is the concentration of agent that inhibits cell growth by 50% compared to the control cells, n is the slope of the curve. In the human melanoma G361 cell line the IC₅₀ of sodium trans-[tetrachlorobis(1H-indazole)ruthenate(III)] (“Test Drug”) was 41 μM (FIG. 1). The G361 cells were relatively resistant to temozolomide (IC₅₀ of temozolomide was 199 μM) (FIG. 2).

The IC₅₀ of sodium trans-[tetrachlorobis(1H-indazole)ruthenate(III)] in the human lung carcinoma A549 cell line was 9.9 μM, highly potent compared to its IC₅₀ in other cell lines. Another human lung carcinoma cell line H1975 was also tested in the same manner as described above to obtain IC₅₀ values of sodium trans-[tetrachlorobis(1H-indazole)ruthenate(III)] and paclitaxel. Paclitaxel is highly effective in H1975 cells (IC₅₀=0.005 μM). Compared to this, A549 cells were relatively resistant to paclitaxel with an IC₅₀ of 9.92 μM. Table 2 below summarizes the activities on the two compounds in the different lung cancer cell lines tested. It is clear that sodium trans-[tetrachlorobis(1H-indazole)ruthenate(III)] (“Test Drug”) is even more effective against lung cancer cells resistant to paclitaxel.

TABLE 2

| | Test Drug | | Paclitaxel | |
|---|---|---|---|---|
| | | IC₅₀ Ratio | | IC₅₀ Ratio |
| NSCLC | IC₅₀ | (A549/H1975) | IC₅₀ | (A549/H1975) |
| A549 | 9.9 μM | 0.29 | 9.92 μM | 1984 |
| H1975 | 33.6 μM | | 0.005 μM | |

In addition, it is known in the art that the human lung carcinoma cell line H1975 is resistant to erlotinib and gefitinib due to the T790M mutation in the EGFR gene in the cells. See e.g., Bao et al., Mol. Cancer Ther., 8(12):3296-3306 (2009). Thus, sodium trans-[tetrachlorobis(1H-indazole)ruthenate(III)] is also active against NSCLC cells resistant to an EGFR inhibitor such as erlotinib and gefitinib, or NSCLC cells having the T790M mutation.

A549 cells are also inherently resistant to gemcitabine. See e.g., Denlinger et al., Ann., Thorac. Surg., 78:1207-1214 (2004). Thus, sodium trans-[tetrachlorobis(1H-indazole)ruthenate(III)] is also active against NSCLC cells resistant to gemcitabine.

3. In Vivo Activities of Sodium Trans-[tetrachlorobis(1H-indazole)ruthenate(III)] in Lung Cancer

A human clinical trial was conducted in two centers in the US with sodium trans-[tetrachlorobis(1H-indazole)ruthenate(III)], which was given intravenously once a week, i.e., on days 1, 8, and 15 of each 28-day cycle to two patients with lung cancer refractory to treatment.

Patient No. 04-005 was a 51 year old white female with Stage IV Non-Small Cell Lung Cancer, moderately differentiated adenocarcinoma histology, diagnosed in April 2006. Initial therapy consisted of radiotherapy (total dose 40 Gy) between May 2006 and June 2006 with a best response of stable disease. Combination chemotherapy with docetaxel, carboplatin and bevacizumab was administered between September and December 2006, with which the patient achieved a complete response. The patient suffered a disease recurrence and was started on sorafenib in April 2008. Sorafenib was discontinued in May 2008 due to disease progression. Therapy was changed to pemetrexed June 2008 through September 2008, with a best response of stable disease. Between November 2009 and January 2010, the patient received gemcitibine. When the patient had disease progression, she was started in March 2010 on sodium trans-[tetrachlorobis(1H-indazole)ruthenate(III)] intravenously at 320 mg/m² (based on body surface area, i.e., BSA) (for a total of 531 mg) once per week on day 1, day 8 and day 15 of each 28-day cycle. The patient received 4 cycles of sodium trans-[tetrachlorobis(1H-indazole)ruthenate(III)] with best response of stable disease. This shows that sodium trans-[tetrachlorobis(1H-indazole)ruthenate(III)] was able to control lung cancer that was refractory to docetaxel, carboplatin, bevacizumab, and pemetrexed, and was resistant to sorafenib and gemcitabine.

Patient No. 04-011 was a 64 year old white male with Stage IIIB Non-Small Cell Lung Cancer, poorly differentiated adenocarcinoma histology, diagnosed in January 2005. Initial therapy consisted of carboplatin, gemcitibine and zoledronic acid from January 2005 through August 2005, with a partial response. Treatment was changed to docetaxel (August 2005 through December 2005) with a best response of stable disease. In December 2005, chest radiotherapy with 54 Gy, with paclitaxel radiosensitization, was initiated and completed by February 2006. Through the remainder of 2006, the patient received a course of single agent erlotinib and a course of single agent pemetrexed; best response to these therapies is unknown. In January 2007, combination therapy with gemcitibine and navelbine was initiated, but his best response was disease progression. Experimental therapy with PTK787 (vatalanib) and imatinib was initiated in February 2007. He remained on that treatment until October 2008 with stable disease. Carboplatin, paclitaxel and bevacizumab therapy was initiated in 2009. The carboplatin and paclitaxel were discontinued in April 2009 and the patient was maintained on bevacizumab until April 2010, when he had disease progression. The patient started therapy with sodium trans-[tetrachlorobis(1H-indazole)ruthenate(III)] in May 2010 as a single agent intravenously at 320 mg/m² (based on body surface area, i.e., BSA) (for a total of 618 mg) weekly on day 1, day 8 and day 15 of each 28-day cycle. The patient received 4 cycles of sodium trans-[tetrachlorobis(1H-indazole)ruthenate(III)] with best response of stable disease. This shows that sodium trans-[tetrachlorobis(1H-indazole)ruthenate(III)] was able to control lung cancer that had been previously treated with carboplatin, gemcitabine, zoledronic acid, erlotinib, pemetrexed, gemcitabine, navelbine, vatalanib, imatinib, carboplatin, paclitaxel and bevacizumab.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. The mere mentioning of the publications and patent applications does not necessarily constitute an admission that they are prior art to the instant application.

Although the foregoing invention has been described in some detail by way of illustration and example for purposes of clarity of understanding, it will be apparent that certain changes and modifications may be practiced within the scope of the appended claims.

We claim:

1. A method of treating lung cancer, comprising:
administering a therapeutically effective amount of an alkali metal salt of trans-[tetrachlorobis(1H-indazole)ruthenate(III)] to a patient having lung cancer that is either (1) identified as having tumor cells harboring the T790M mutation in the EGFR gene, or (2) previously treated with a regimen comprising one or more drugs chosen from the group of paclitaxel, docetaxel, carboplatin, bevacizumab, sorafenib, gemcitabine, zoledronic acid, pemetrexed, navelbine, vatalanib, imatinib, erlotinib, gefitinib, cetuximab and panutimumab.

2. The method of claim 1, wherein the lung cancer is NSCLC.

3. The method of claim 1, wherein sodium trans-[tetrachlorobis(1H-indazole)ruthenate(III)] is administered.

4. The method of claim 1, wherein sodium trans-[tetrachlorobis(1H-indazole)ruthenate(III)] is administered intravenously at an amount of at least 320 mg/m².

5. The method of claim 1, wherein sodium trans-[tetrachlorobis(1H-indazole)ruthenate(III)] is administered intravenously at an amount of at least 500 mg/m².

6. The method of claim 1, wherein the lung cancer has previously been treated with a regimen comprising paclitaxel.

7. The method of claim 1, wherein the lung cancer has previously been treated with a regimen comprising gemcitabine.

8. The method of claim 1, wherein the lung cancer has previously been treated with a regimen comprising erlotinib or gefitinib.

9. The method of claim 1, wherein the lung cancer has previously been treated with a regimen comprising sorafenib.

10. The method of claim 1, wherein the lung cancer has previously been treated with a regimen comprising bevacizumab.

11. The method of claim 1, wherein the lung cancer has previously been treated with a regimen comprising docetaxel and/or carboplatin.
